(19) United States
(12) Patent Application Publication
(10) Pub. No.: US 2006/0161149 A1
(43) Pub. Date: Jul. 20, 2006

(54) Surgical ablation device

(76) Inventors: Salvatore Privitera, Mason, OH (US); Keith Edward Martin, Cincinnati, OH (US); Michael Dawson Hooven, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 11/037,543

(22) Filed: Jan. 18, 2005

(51) Int. Cl. A61B 18/14

(52) U.S. Cl. 606/41

(57) ABSTRACT

A surgical ablation device. A pair of electrodes, each electrode having a surface area for contacting tissue. A pair of heat sinks each in thermal communication with an electrode, the heat sinks having a volume such that the ratio of surface area of the electrode to volume of the heat sink is less than about 3 in²/in³. The electrodes may be slender and parallel. When the electrodes are energized with bi-polar electric energy and placed in contact with the tissue surface, the tissue is heated and ablated such that the maximum tissue temperature occurs below the tissue surface.

### BACKGROUND

The present invention relates to surgical instruments, with examples relating to bi-polar ablation devices and a systems for controlling such devices. Surgery generally refers to the diagnosis or treatment of injury, deformity, or disease. In a variety of surgical procedures, it is desired to ablated tissue or cause lesions in tissue. Some examples of such procedures include, without limitation, electrical isolation of the pulmonary veins to treat atrial fibrillation, ablation of uterine tissue associated with endometriosis, ablation of esophageal tissue associated with Barrett's esophagus, ablation of cancerous liver tissue, and the like. The foregoing examples are merely illustrative and not exhaustive. While a variety of techniques and devices have been used to ablate or cause lesions in tissue, no one has previously made or used an ablation device in accordance with the present invention.

### BRIEF DESCRIPTION OF DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 illustrates a perspective view of an example of an ablation device;

FIG. 2 illustrates a perspective detailed view of the head of the ablation device of FIG. 1;

FIG. 3 illustrates an exploded view of the head of the ablation device of FIG. 1;

FIG. 4 illustrates a cross-sectional view of the head of the ablation device of FIG. 1;

FIG. 5 illustrates a perspective view of an example of an ablation device with a roller head;

FIG. 6 illustrates a perspective detailed view of the roller head of the ablation device of FIG. 5;

FIG. 7 illustrates an exploded view of the roller head of the ablation device of FIG. 5;

FIG. 8 illustrates a cross-sectional view of the roller head of the ablation device of FIG. 5;

FIG. 9 illustrates an example of temperature gradients in tissue;

FIG. 10 illustrates an example of a power output curve for an ablation device; and

FIG. 11 illustrates an example of potential and current curves for an ablation device.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

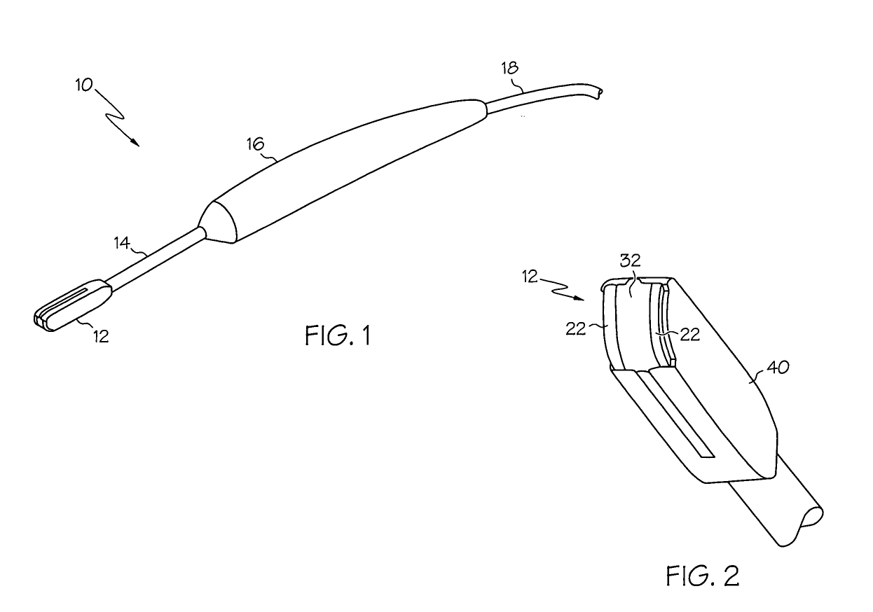

FIG. 1 illustrates an example of an ablation device (10). The ablation device (10) in this embodiment is a handheld wand. The ablation device (10) includes a head (12) connected to the distal end of a shaft (14), and a handle (16) connected to the proximal end of the shaft (14). As shown here, the shaft (14) is straight and substantially rigid; however, flexible, curved, malleable or articulated shafts could also be used depending on the surgical procedure or anatomy being treated. A power source (not shown) is connected to the cord (18).

FIG. 2 illustrates an more detailed view of the head (12) of the ablation device (10). The head (12) includes two electrodes (22), which are capable of being energized with bi-polar energy. In the present example, each electrode (22) includes a smooth surface area for contacting tissue. Each electrode (22) is slender in the sense that the length of the tissue contacting surface is at least 4 times its width. As shown in the present example, the length is between about 5 to 7 times the width. The electrodes (22) in this example are substantially parallel to one another, and as shown here the electrodes (22) are spaced between about 2 to 4 mm from one another. An electrically insulative surface (32) is interposed between the electrodes (22). In this example, the surface (32) is convex between the electrodes (22), distally extending about 0.01 inches from the lateral plane between the electrodes (22). As shown in the figures, a portion of the distal tip of the head (12) curved along the transverse axis. In the present example the curved end is an arc with a radius between 0.19 and 0.21 inches. The electrodes (22) and surface (32) have similar curves. An electrically insulative sheath (40) covers other portions of the head (12).

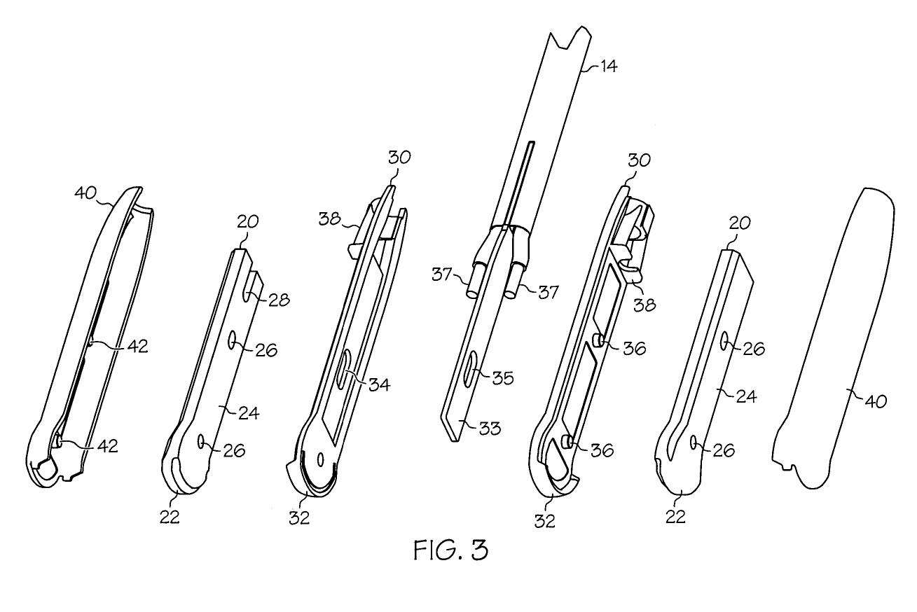

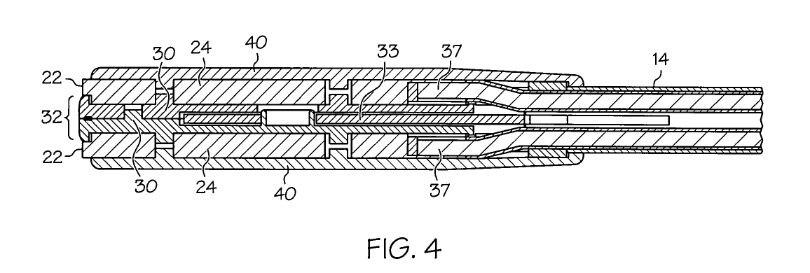

FIGS. 3 and 4 illustrate the component parts of the head (12) and some related structures. A rib (33) extends distally from the shaft (14). Electrical wires in communication with the cord (18) pass through the shaft (14) and end with electrical terminals (37). A pair of electrical insulators (30) laterally connect to either side of the rib (33). The distal tips of the insulators (30) define the insulative surface (32). A post (hidden in this view) on the right insulator (30) mates with the holes (35, 34). A receiving structure (38) is dimensioned to hold the terminals (37) in their desired positions.

Two conductors (20) laterally connect with the insulators (30). In the present example, each conductor (20) is a contiguous and unitary part; however, two or more components could form the conductor (20). Also in this example, each conductor (20) is a homogeneous material. Each conductor (20) includes an electrode (22) and heat sink (24). Each conductor has a recess (28) dimensioned to snugly receive the corresponding terminal (37), thus facilitating electrical contact with the terminal (37). The sheath (40) covers the assembled head (12). Posts (42, 36) mate with the holes (26) in the conductor (20) to facilitate and maintain alignment of the assembly. The distal ends of the conductors (20), bounded by the surface (32) and the sheath (40), define the surface areas of the electrodes (22).

The conductor (20) in this example is electrically conductive, thus facilitating the flow of current from the terminal (37) to the electrode (22). The conductor (20) in this example is also thermally conductive, thus facilitating the flow of heat from the electrode (22) to the heat sink (24). Some suitable materials for the conductor (22) include, without limitation, copper, silver, gold, platinum, titanium, aluminum, beryllium, nickel, and the like. In one variation, the heat sink (24) is copper while the electrode (22) is gold plated. The heat sink (24) has a volume, which in this example is the volume of the conductor (20). Preferably, the ratio of tissue contacting surface area of the electrode (22) to volume of the heat sink (24) is less than about 3 in²/in³. In the present example, the ratio is less than about 1 in²/in³.

One illustrative use of the device (10) is during surgery to ablate tissue. The surface area of the electrodes (22) are placed in contact with the tissue surface. The electrodes (22) are energized with bi-polar energy by connecting the device (10) to an electric power source. As one with ordinary skill in the art will readily appreciate, RF energy is transmitted to the tissue through the electrodes (22), thus heating the tissue until ablated and the desired lesion is formed in the tissue. Optionally, the head (12) can be swiped over the tissue surface, either laterally or transversely, while maintaining the electrodes (22) in contact with the tissue to ablate larger areas or to ablate the tissue in a desired pattern. The heat sink (24) draws heat away from the tissue during the ablation process, thus reducing the temperature elevation of the tissue surface. The temperature reduction has the benefit (among other benefits) of facilitating deeper and more controlled lesions, including, when desired, transmural lesions through a tissue wall.

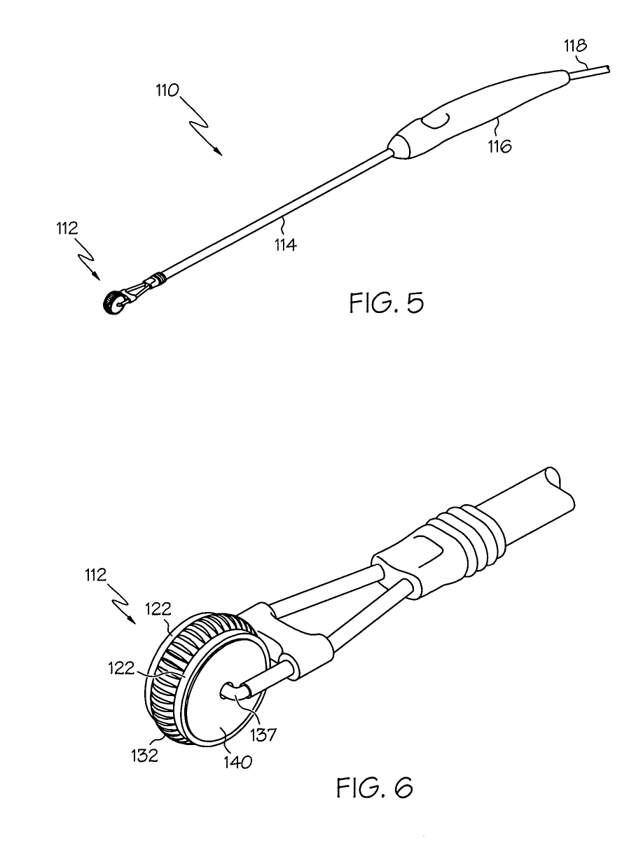

FIG. 5 illustrates another example of an ablation device (110). The ablation device (110) in this embodiment is a handheld wand. The ablation device (110) includes a roller head (112) connected to the distal end of a shaft (114), and a handle (116) connected to the proximal end of the shaft (114). As shown here, the shaft (114) is straight and substantially rigid; however, flexible, curved, malleable, or articulated shafts could also be used depending on the surgical procedure or anatomy being treated. A power source (not shown) is connected to the cord (118).

FIG. 6 illustrates an more detailed view of the roller head (112) of the ablation device (110). The roller head (112) in this example rotates about the axis between the terminals (137). The roller head (112) includes two electrodes (122), which are capable of being energized with bi-polar energy. In the present example, each electrode (122) includes an smooth surface area for contacting tissue. In one embodiment, the diameter of the electrodes (122) is between about 10 mm and about 20 mm. Each electrode (122) is slender, and as shown in the present example the length of tissue contacting surface is between about 5 to 7 times width assuming a 60 degree contact with tissue, or alternatively a circumferential length of between about 30-42 times the width. The electrodes (122) in this example are substantially parallel to one another around the circumference of the roller head (112), and as shown here the electrodes (122) are spaced between about 2 to 4 mm from one another. The electrodes (122) are perpendicular to the axis of rotation of the roller head (112). An electrically insulative surface (132) is interposed between the electrodes (122). In this example, the surface (132) is convex between the electrodes (22), radially extending about 0.01 inches from the lateral plane between the electrodes (122). Optionally, the surface (132) includes a tread to improve traction with the tissue being treated. In the present example, the tread takes the form of lateral grooves; however, other tread patterns could be used. An electrically insulative sheath (140) covers the lateral faces of the roller head (112).

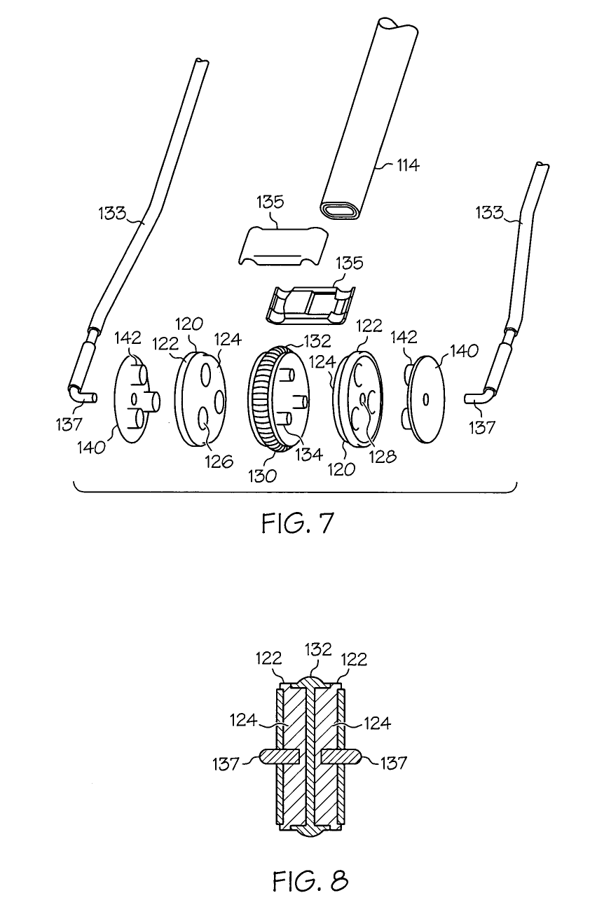

FIGS. 7 and 8 illustrate the component parts of the roller head (112) and some related structures. A pair of struts (133) are positioned in the shaft (114). Each strut (133) includes an electrically conductive shaft covered in an electrical insulator, and is in electrical communication with the cord (118). A terminal (137) is positioned at the distal end of each strut (133). A brace (135) is connected to the struts (133) and facilitates alignment and structural integrity of the assembly. Optionally, a fender (not shown) may be attached to the brace and cover a circumferential portion of the roller head (112). An electrical insulator (130) is positioned in the center of the roller head (112). Two circular conductors (120) laterally connect on either side of the insulator (130). In the present example, each conductor (120) is a contiguous and unitary part; however, two or more components could form the conductor (120). Also in this example, each conductor (120) is a homogeneous material. Each conductor (120) includes an electrode (122) and heat sink (124). A recess (128) is provided in the center of the conductor (122) and is dimensioned to receive the corresponding terminal (137). The terminal (137) functions as an axle, thus allowing the roller head (112) to rotate. The interface between the terminal (122) and recess (128) allows sufficient contact to permit an electrical connection between the conductor (120) and the terminal (137). A sheath (140) laterally connects to each conductor (120). Posts (142, 136) mate with the holes (126) in the conductor (120) to maintain alignment of the assembly. The radial ends of the conductors (120), bounded by the surface (132) and the sheath (140), define the surface areas of the electrodes (122).

The conductor (120) in this example is electrically conductive, thus facilitating the flow of current from the terminal (137) to the electrode (122). The conductor (120) in this example is also thermally conductive, thus facilitating the flow of heat from the electrode (122) to the heat sink (124). The conductor (120) may be made from similar materials as the conductor (20) disclosed above. The heat sink (124) has a volume, which in this example is the volume of the conductor (120). Preferably, of surface area of the electrode (122) and volume of the heat sink (124) have a similar ratio as the conductor (20) disclosed above. Only a portion of the circumference (e.g. about 60 degrees) of the electrodes (122) will be in contact with tissue during use, so only the tissue contacting portion should be used in making the ratio calculation.

One illustrative use of the device (110) is during surgery to ablate tissue. The electrodes (122) are placed in contact with the tissue surface. The electrodes (122) are energized with bi-polar energy by connecting the device (110) to an electric power source. As one with ordinary skill in the art will readily appreciate, RF energy is transmitted to the tissue through the electrodes (122), thus heating the tissue until ablated and the desired lesion is formed in the tissue. The head (12) may be rolled over tissue while maintaining the electrodes (122) in contact with the tissue to ablate larger areas or ablate the tissue in a desired pattern. The heat sink (124) draws heat away from the tissue during the ablation process, thus reducing the temperature of the tissue.

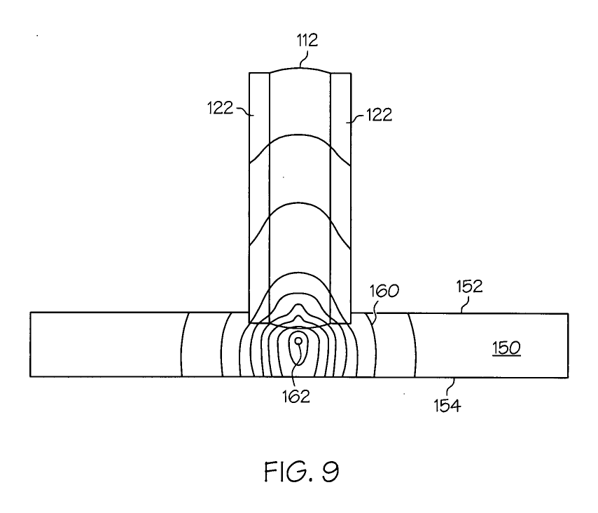

FIG. 9 illustrates an example of the temperature gradients when the roller head (112) is used. It should be apparent that similar gradients will be experienced when the head (12) is used. The tissue (150) being treated includes a proximal side (152) and a distal side (154). In use, the roller head (112) is placed onto the proximal side (152) of the tissue. The isothermal lines (160) illustrate the temperature distribution in the tissue (150) and demonstrate the heat absorption by the heat sink (not shown). The maximum tissue temperature (162) occurs inside the tissue wall, below the tissue surfaces (152, 154).

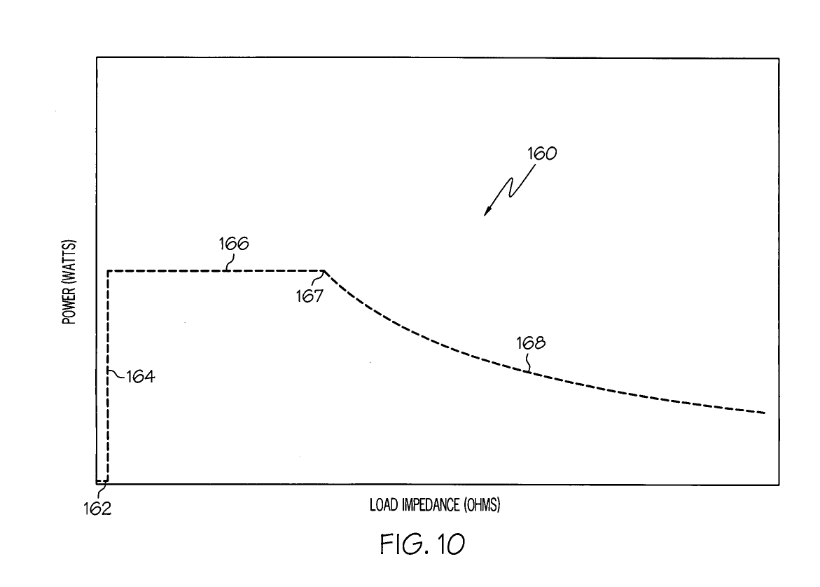

FIG. 10 illustrates an example of a power output curve (160) for a bi-polar ablation device. While the power output curve (160) is very suitable for use with the devices (10, 110) disclosed above, it could also be used with other bi-polar ablation devices, including without limitation bi-polar clamp devices such as those disclosed in U.S. Pat. No. 6,517,536. The x-axis represents the load impedance of the tissue being treated, and the y-axis represents the power output by the bi-polar device into the tissue. The load impedance can be measured between the electrodes of the bi-polar device. As one with ordinary skill in the art will readily recognized, a feedback control system (located in the device or the power source) can be used to energize the electrodes and adjust the power output in real-time based on the measured load impedance.

In the present example, the power output (162) is zero or near zero below a first threshold impedance indicating an electrical short or other problem with the ablation device. The first threshold impedance may be less than about 60 ohms, but as shown in the present example the first threshold impedance is less than about 20 ohms. At or above this first threshold, the power raised (164) to an operating power output (166). In the present example, the operating power output (166) may be maintained at a substantially constant wattage level between 10-20 watts. The output wattage may vary based on a number of criteria. For instance, in one embodiment the operating power output (166) could be substantially constant at about 15 watts, while in anther embodiment the operating power output (166) could be about 18 watts. After a second threshold impedance (167), the electrodes are energized to produce a variable power output (168) inversely related to the load impedance. The second threshold impedance (167) may vary based on a number of criteria. For instance, the second threshold impedance may be between 250-500 ohms. In one embodiment, the second threshold impedance is about 400 ohms. The variable power output (168) may be adjusted as part of a feedback control logic based on the measured tissue impedance, adjusted as a function of time, or adjusted as part of a feedback control logic based on the measured tissue temperature. In one embodiment, variable power output (168) continues energizing the electrodes until a transmural lesion is produced in the tissue wall.

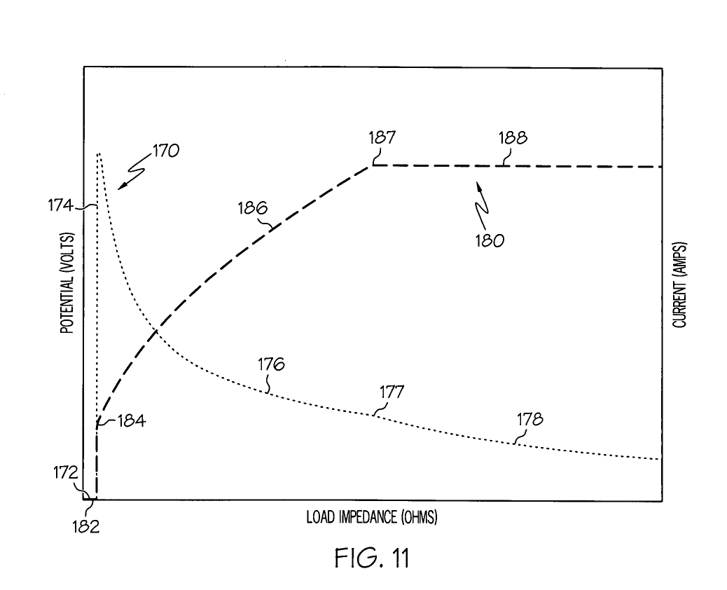

FIG. 11 illustrates two of many possible control curves to produced the power output curve (160). As one with ordinary skill in the art will readily recognize, power is a function of potential and current. Thus, current and potential from a power source can be adjusted in accordance with the respective curves (170, 180) to produce the power output curve (160). The x-axis represents the load impedance of the tissue, and the y-axes represent potential and current being delivered to the bi-polar electrodes of the ablation device. The current (172) is zero or near zero below the first threshold impedance. The current is raised (174) at or above this first threshold, and a variable current (176) is delivered inversely related to the load impedance. At or above the second threshold impedance (177), the variable current pattern (178) may be modified while still relating inversely to the load impedance. The potential (182) is zero or near zero below the first threshold impedance. The potential is raised (184) at or above this first threshold, and a variable potential (186) is delivered as a function of the load impedance up to the second threshold impedance (187). At or above the second threshold impedance (187), a substantially constant potential (188) is delivered.

The power output curve (160) represents only one example of such a curve and a variety of other curves for patterns could also be used. As indicated above, the power output curve (160) may also vary based on number of criteria for a particular surgical procedure. Without limitation, three such criteria include the type of tissue being treated, the thickness of the tissue, and the depth of the desired lesion. The criteria could be input in a number of ways. For instance, the operator could select from two or more the power output curves on the power source. Alternatively, the operator may program the power source to match a custom power output curve. Optionally, a given ablation device (e.g., wand devices, a bi-polar clamps, or others) may be designated for a particular type of surgical procedure. For instance, one bi-polar clamp could be designated for treatment of cardiac tissue, while a bi-polar wand could be designated for treatment of liver tissue. Each device could be configured to have a unique code so that when connected to the power source, the power source would recognize the code and automatically select the power output curve corresponding to the ablation device.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A surgical device for ablating tissue using electrical energy, comprising:
a) a pair of electrodes, each electrode having a surface area for contacting tissue; and
b) a pair of heat sinks each in thermal communication with an electrode, the heat sinks having a volume such that the ratio of surface area of the electrode to volume of the heat sink is less than about 3 in²/in³.

2. The surgical device of claim 1, wherein the electrode and corresponding heat sink are a homogenous material.

3. The surgical device of claim 1, wherein electrode and corresponding heat sink are contiguous.

4. The surgical device of claim 1, wherein the electrodes are slender.

5. The surgical device of claim 4, wherein the slender electrodes are parallel.

6. The surgical device of claim 5, wherein the electrodes are spaced apart between about 2 and about 4 mm.

7. The surgical device of claim 1, wherein the electrodes and heat sinks are a roller head.

8. The surgical device of claim 7, wherein the roller has a diameter between about 10 mm and about 20 mm.

9. The surgical device of claim 1, wherein the electrodes are smooth.

10. The surgical device of claim 1, further comprising an electrically insulative surface interposed between the pair of electrodes.

11. The surgical device of claim 10, wherein the insulative surface has a convex surface extending between the pair of electrodes.

12. The surgical device of claim 1, wherein the electrodes are bi-polar.

13. A method for ablating tissue using the device of claim 1, the method comprising:
a) placing the pair of electrodes in contact with the surface of tissue;
b) connecting the electrodes to an electric power source;
c) transmitting RF energy to the tissue through the electrodes; and
d) heating the tissue with the RF energy until the tissue is ablated.

14. The surgical device of claim 13, wherein the maximum tissue temperature is below the tissue surface.

15. A surgical device for treatment of tissue, the tissue having a tissue surface, comprising:
a) a pair of electrodes having a surface area for contacting the tissue surface;
b) a heat sink thermally coupled to the electrodes;
whereby when the electrodes are energized with bi-polar electric energy and placed in contact with the tissue surface, the tissue is heated and ablated such that the maximum tissue temperature occurs below the tissue surface.

16. The surgical device of claim 15, wherein the electrodes are slender and parallel.

17. The surgical device of claim 15, wherein each electrode has a corresponding heat sink, and each electrode and heat sink is a contiguous and unitary part.

18. The surgical device of claim 15, further comprising an electrically resistant surface interposed between the electrodes, the body having a convex surface extending between the pair of electrodes.

19. The surgical device of claim 15, wherein heat sinks having a volume such that the ratio of surface area of the electrode to volume of the heat sink is less than about 3 in²/in³.

20. A bi-polar surgical device, comprising:
a) a first conductor comprising a heat sink and an electrode in a unitary part, the electrode being slender and having a tissue contacting surface area, wherein the heat sink has a volume such that the ratio of surface area of the electrode to volume of the heat sink is less than about 3 in²/in³;
b) a second conductor comprising a heat sink and an electrode in a unitary part, the electrode being slender, having a tissue contacting surface area, and being parallel to the electrode of the first conductor, wherein the heat sink has a volume such that the ratio of surface area of the electrode to volume of the heat sink is less than about 3 in²/in³;
c) an insulator interposed between the first and second conductors, the insulator comprising a convex surface interposed between the electrodes; and
d) a power source for energizing the electrodes with bi-polar electrical energy.
